(19) United States
(12) Patent Application Publication
(10) Pub. No.: US 2009/0214658 A1
(43) Pub. Date: Aug. 27, 2009

(54) Inorganic sorbent copolymer

(76) Inventors: Gritsenko Anatoly Grigor'evich, Moscow (RU); Bratislav Jovanovic, San Diego, CA (US)

Correspondence Address: CHESTER G. MOORE;Baker, Donelson, Bearman, Caldwell & Berkowitz, PC, 3 Sanctuary Boulevard, SUITE 201, Mandeville, LA, 70471, US

(21) Appl. No.: 12/100,734

(22) Filed: Apr. 10, 2008

Related U.S. Application Data
(60) Provisional application No. 61/030,648, filed on Feb. 22, 2008.

(51) Int. Cl. A61K 33/24; A61K 9/14; A61P 31/12; A61P 31/18

(52) U.S. Cl. 424/489; 424/651

(57) ABSTRACT

A medicinal preparation useful for treating viral diseases and for prophylaxis of various diseases—including zoonotic diseases—along with methods of using the same, is described. The preparation comprises a fine crystalline antimony silicate-based sorbent of the compositional formula xA₂O.ySb₂O₅.zSiO₂, wherein x is between 0.5 and 3, y is between 0.5 and 1.5, z is between 0.5 and 4; and A is H, Na, K, Rb, Cs. The sorbent particles are between 0.1 and 100 μm in diameter. Between 100 and 120 mg/kg of sorbent may be administered intravenously, intramuscularly, or orally (e.g., via capsules).

### CROSS-REFERENCE TO RELATED APPLICATIONS

This Non-Provisional patent application, filed under 35 U.S.C. § 111 (a), claims the benefit under 35 U.S.C. § 119(e)(1) of U.S. Provisional Patent Application No. 61/830,648, filed under 35 U.S.C. § 111(b) on 22 Feb. 2008, and which is hereby incorporated by reference in its entirety.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

Not applicable.

### BACKGROUND OF THE INVENTION

1. Field of the Invention

The present invention provides a method of treatment and prophylaxis for viral diseases, comprising an inorganic, antimony-containing compound. More specifically, the invention provides a method of treating viral diseases by administering to a subject in need thereof a material comprising antimony silicates with the compositional formula:

xA₂O.ySb₂O₅.zSiO₂

wherein x is between 0.5 and 3, y is between 0.5 and 1.5, z is between 0.5 and 4; and A is Na, K, Rb, or Cs.

2. Description of Related Art

Porous, antimony-containing inorganic sorbents in a solution of monosubsitituted sodium phosphate and sodium metasilicate are known (see, e.g., Ogenko et al., SU 1156728 A1). However, these sorbents are inappropriate for the treatment of viral diseases.

An antiviral agent containing antimony has been described previously. An injectable compound of complexed heteropolyanions containing tungsten combined with antimony was described as a medicament for the curative and preventive treatment of diverse viral infections (see Chemann et al., U.S. Pat. No. 4,547,369). The compound of Chemann et al. is a copolymer of trivalent antimony Sb(III) and sodium tungstate, with the number of antimony atoms being in a 2.5:1 ratio to the number of atoms of tungsten. Although activity against leukemia viruses, sarcoma viruses, M-MSV virus, Friend virus (plasma variant), sarcomatogenic viruses, encephalomyocardial virus, and rabies virus is claimed for this preparation, the compound is ineffective for the treatment of many common veterinary viral diseases including but not limited to Aujeszky's disease (also called “pseudorabies,” a viral disease due to porcine herpesvirus-1, which belongs to the Alphaherpesvirinae subfamily, Herpesviridae family) in rabbits, rotavirus infection of cattle, and Newcastle disease in birds. Furthermore, the preparation of Chemann et al. cannot ensure elimination of viral toxins or toxic viral byproducts.

### BRIEF SUMMARY OF THE INVENTION

The present invention provides methods of treating viral diseases comprising administering to a subject in need thereof a compound of Formula 1:

xA₂O.ySb₂O₅.zSiO₂ (Formula 1)

wherein x is between 0.5 and 3, y is between 0.5 and 1.5, z is between 0.5 and 4; and A is Na, K, Rb, Cs.

The compound of Formula 1 is an ion exchange sorbent material useful for treatment of infections in mammals and birds, including but not limited to Aujeszky's disease, rotavirus infection, Newcastle disease, viral hepatitis, and leucosis.

The present invention provides methods of treating an infection, the method comprising administering to a mammal in need thereof a compound of the formula xA₂O.ySb₂O₅.zSiO₂, wherein x is between 0.5 and 3, y is between 0.5 and 1.5, z is between 0.5 and 4; and A is H, Na, K, Rb, or Cs, and wherein the compound has a particle size range, said particle size range being between 0.1 and 100 μm.

In one favored aspect of the invention, the route of administration is oral. In another favored aspect of the invention, the route of administration is intramuscular. The infection to be treated may be a viral infection, and said viral infection may be Aujeszky's disease, and the viral infection may also be caused by rabies virus, rotavirus, bovine leucosis virus, hepatitis C virus, influenza virus (particularly Influenza A virus subtype H5N1), and a human immunodeficiency virus.

### DEFINITIONS

The following definitions are provided to give a clear understanding of the specification and appended claims.

As used herein, the term “sorbent” means a material having the property of collecting molecules of a substance by sorption. The term also refers to that which sorbs, and to things having this property (e.g., having a high sorbent capacity).

As used herein, the term “molarity,” “molar,” or “M” denotes the number of moles (mol) of a given substance per liter (L) of solution (e.g., M=mol/L). For example, a solution containing 2.0 moles of dissolved particles (solute) in a 4.0 L solution is a 0.5 molar solution. It should be noted that such solution does not contain 4.0 L of solvent. Such solution will contain slightly more or less than 4.0 L of solvent because the process of dissolving the solute causes the total volume to increase or decrease.

As used herein, the acronym “mEq” refers to a “milliequivalent,” which is a measure of a substance's ability to combine with other substances. Formally, an “equivalent” (Eq) is defined as the mass (in grams) of a substance that will react with a mole (6.022×10²³) of electrons (e). A milliequivalent is one one-thousandth of an equivalent (Eq 1,000). For monovalent ions, 1 Eq is equal to 1 mol. For divalent ions, 1 Eq is equal to 0.5 mol. Ion exchange capacity is usually expressed in terms of equivalents per liter (Eq/L) or equivalents per gram (Eq/g) of material. An equivalent is the molecular weight—in grams—of the compound, divided by its electrical charge (its valence). For example, a material an exchange capacity of 1 Eq/L could remove 32.7 g of divalent zinc (Zn²⁺; molecular weight of 65.38 g/mol) from a 1 L solution.

As used herein, “CAS No.” means “Chemical Abstracts Service number.”

As used herein, “L” means “liter”; “ml” means “milliliter”; “μl” means “microliter”; “g” means “gram”; “kg” means “kilogram”; “hr” means “hour”; “p.o.” or “PO” means “per os,” or “orally” and indicates administration by mouth; “i.m.” or “IM” means “intramuscular,” and indicates administration by injection into muscle tissue; “i.v.” or “IV” means “intravenous,” and indicates administration by intravenous injection.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements.

FIG. 1 illustrates—on a log-linear scale—the mean concentration over time of sorbent copolymer in rabbit blood, following intravenous (filled circles) and oral (open circles) administration of 12% sorbent copolymer.

FIG. 2 illustrates—on a log-linear scale—the mean concentration over time of sorbent copolymer in dog blood, following a single intravenous injection of 12% sorbent copolymer in water.

FIG. 3 illustrates—on a log-linear scale—the mean concentration over time of sorbent copolymer in dog blood, following a single intramuscular injection of 12% sorbent copolymer in water.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

An object of the present invention is to provide a preparation for treatment of viral diseases, and for the prophylaxis of infectious epizootic processes. This goal is realized with a material comprising antimony silicate that is obtained by the simultaneous polymerization of the hydrolysis products of antimony oxytrichloride (SbOCl₃) or antimony pentachloride (SbCl₅; CAS No. 7-647-18-9) with sodium metasilicate pentahydrate (Na₂SiO₃.5H₂O; CAS No. 10213-79-3) or sodium trisilicate (Na₂Si₃O₇; CAS No 304671-98-5) in acidic aqueous media. The prepared silicate is a fine crystalline material with the compositional formula of xA₂O.ySb₂O₅.zSiO₂, wherein x is between 0.5 and 3, y is between 0.5 and 1.5, z is between 0.5 and 4; and A is Na, K, Rb, or Cs, having a particle diameter of between 0.1 and 100 μm (microns), and provided to a mammal in need thereof in a dose range between 100 and 120 mg per kg (kilogram) of body weight. The preparation contains 47 to 55% antimony, bound by silicon dioxide. The silicon content is preferably 4 to 4.7%. The pure product may contain, as byproducts of synthesis, sodium nitrate (NaNO₃; less than 0.1%), sodium chloride (NaCl; less than 1%), sodium phosphates (for example: sodium monophosphate NaH₂PO₄; trace amount—i.e., less than 0.001%), antimony trioxide not bound in the product compound (Sb₂O₃; trace amount), and antimony pentoxide (Sb₂O₃; trace amount).

The active sorbent substance is a fine, white crystalline powder. It has no odor, is slightly acidic, and the pH of aqueous dispersion is between 5.0-6.5. The particles are between 0.1 and 100 μm in diameter, and are virtually insoluble in water, 95% ethanol, diethyl ether, benzene, or acetone. Neither is it soluble in sulfuric or nitric acids. In twice-distilled water it forms a milk-white suspension. The sorbent can be obtained, for example, by a method described in RU 2143315, 1999. The raw material used to prepare the sorbent is antimony trioxide (Sb₂O₃; CAS No. 1309-64-4) or antimony trichloride (SbCl₃; CAS No. 10025-91-9), sodium metasilicate (Na₂SiO₃; CAS No. 10213-79-3), and monobasic sodium phosphate dihydrate (NaH₂PO₄.2H₂O; CAS No. 13472-35-0). The final antiviral preparation is obtained, after chemical synthesis, by grinding the sorbent copolymer particles until the particle diameters are between 0.1 and 100 μm, and preferably between 0.1 and 20 μm.

The material of the present invention, a highly effective sorbent, suppresses the infectious activity of a variety of viruses by blocking the initial stages of viral replication in cells infected by the virus. The prepared material, through its sorbent properties, also neutralizes thermostable and thermolabile protein toxins. Because the finer formulation comprising the material of the present invention may also be injected intravenously, it can be used to rapidly bind blood-borne endotoxins, exotoxins, and products of incomplete toxin breakdown, thus facilitating their elimination from the body. The preparation may additionally contain water as a dosing vehicle, and may be provided either as a suspension or as a capsule filled with the suspension.

Pharmacological Properties

The sorbent material of the present invention is nontoxic, it has no cumulative properties (i.e., the copolymer is eliminated from the body completely, via urinary and biliary excretion), and does not exhibit fetotoxic, teratogenic, or mutagenic action. The copolymer of the present invention may be administered orally, intramuscularly, and intravenously in doses of 100 to 120 mg/kg therapeutically and prophylactically in animals with viral infections, especially mammals, and most especially humans.

As an ion-exchange material, the sorbent material of the present invention exerts a unique effect on viral infection in the body, and with its bioavailability and extra-small particle size, given its mean particle size of between 0.1 and 100 microns, it penetrates into the body's organs and tissues. The surfaces of the polysilicate particles are easily accessible for sorbtion of molecules of any size. The polysilicate possesses excellent hydrophilicity, is easily wetted by water and forming a suspension with it, and has high sorption capability. When a 10 mg/kg bolus is administered intravenously, the maximum concentration in blood is reached during 1 hour and persists for up to 72 hours. When administered at 100 mg/kg by oral gavage, maximum concentration is reached after 72 hours. The copolymer is also observed to have well-balanced osmotic and hydrophilic properties, which are responsible for its antiviral action, and it is eliminated from the body via the renal, hepatic, and gastrointestinal systems.

Pharmacokinetic Properties

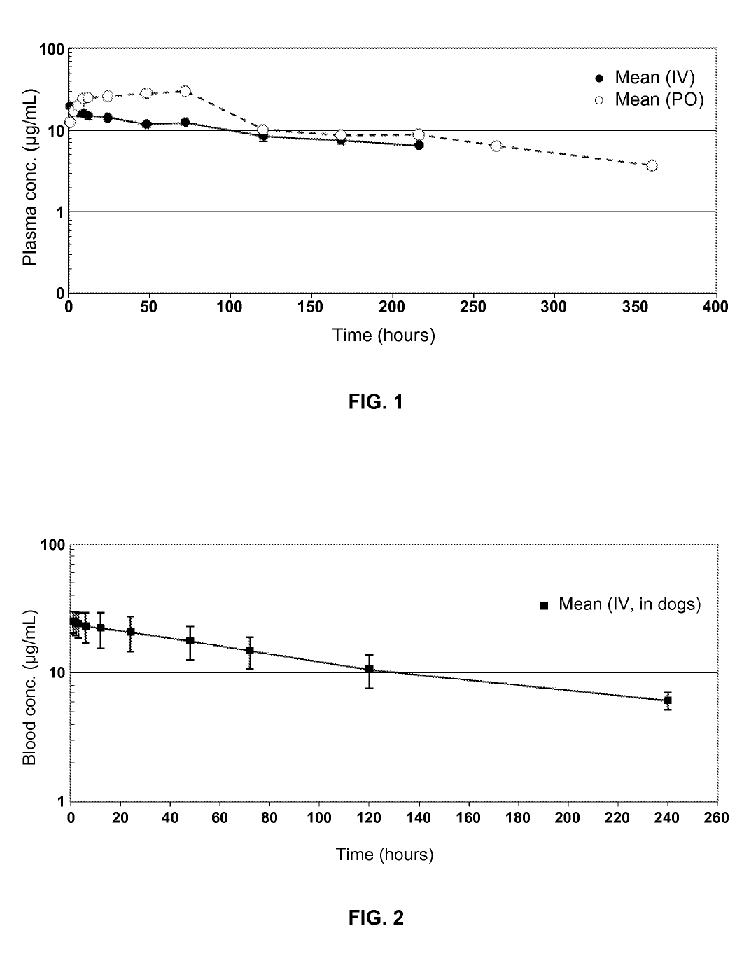

A pharmacokinetic study was conducted using fasting rabbits (n=6; average weight of 4 kg) to determine the time course of sorbent copolymer disposition following single intravenous bolus or oral gavage administrations of sorbent copolymer (10% suspension in water) at 10 and 100 mg/kg, respectively. Regular sampling of whole blood was performed throughout a 360 hour period after dose administration, and key pharmacokinetic parameters were derived. For animals treated intravenously, blood was sampled at 1, 3, 9, 12, 24, 48, 72, 120, 168, and 216 hours after administration; for animals treated by oral gavage, blood was drawn at the same time points and also at 360 hours. FIG. 1 illustrates—on a log-linear scale—the mean concentration over time of sorbent copolymer in rabbit blood, following intravenous (filled circles) and oral (open circles) administration of 12% sorbent copolymer.

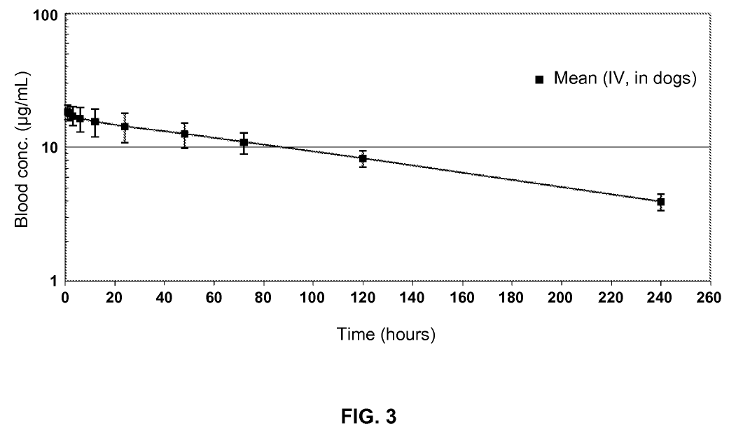

In addition to the rabbit pharmacokinetic studies, a preliminary pharmacokinetic study using fasting dogs (=6; average weight of 12.65 kg) was also conducted. Either a single intravenous bolus or an intramuscular injection of sorbent copolymer (12% in water) was administered at 100 mg/kg. Blood was drawn at 1, 2, 3, 6, 12, 24, 48, 72, 120, and 240 hours after sorbent copolymer administration, and key pharmacokinetic parameters were derived. FIGS. 2 and 3 illustrate—on a log-linear scale—the mean concentration over time of sorbent copolymer in dog blood, following a single intravenous or a single intramuscular injection of 12% sorbent copolymer in water, respectively. A comparison of the key pharmacokinetic parameters from both rabbits and dogs is listed in TABLE 1.

TABLE 1
Comparison of rabbit and dog sorbent copolymer pharmacokinetic data

| | | | Dogs | |
|---|---|---|---|---|
| Pharmacokinetic Parameter | Rabbits | | IV, | |
| (units) | IV, bolus | PO, gavage | bolus | IM |
| Dose (mg/kg) | 10 | 100 | 100 | 100 |
| Cₘₐₓ (mg/mL) | 19.4 | 30 | 25.1 | 18.4 |
| Tₘₐₓ (hr) | N/A | 72 | N/A | N/A |
| Cₘᵢₙ (mg/mL) | 6.5 | 3.7 | 6.14 | 3.9 |
| AUC_{(0-240 hr)} (μg · hr/mL) | 2188.7 | N/A | 3025.5 | 2169.9 |
| AUC_{(0-360 hr)} (mg · hr/mL) | N/A | 4559.4 | N/A | N/A |
| Cls (L/kg/hr) | 2.9 | N/A | 2.5 | N/A |
| Vd (L/kg) | 5.7 | 3.4 | 4.05 | 5.84 |
| T_{1/2} (hr) | 100.4 | 117.5 | 118 | 110.8 |
| F (%) | N/A | 20.9 | N/A | N/A |

Cₘₐₓ = “peak concentration”;
Tₘₐₓ = “time to reach peak concentration”;
Cₘᵢₙ = “trough concentration”;
AUC = “area under the curve”;
Cls = “clearance”;
Vd = “volume of distribution”;
T_{1/2} = “half-life”;
F = “bioavailability”.

The copolymer preparation does not evoke a pyrogenic reaction, it does not suppress immunity, and it is highly effective whether administered orally or intravenously. At therapeutic doses, the copolymer exhibits high activity, even at the peak of the diseases cited above. The copolymer is useful for treatment of Aujeszky's disease (also called “pseudorabies,” a viral disease due to porcine herpesvirus-1, which belongs to the Alphaherpesvirinae subfamily, Herpesviridae family) in rabbits, rotavirus infection of cattle, and Newcastle disease in birds. The copolymer may also be used with excellent results for prophylactic purposes for many infectious diseases, including but not limited to those caused by hepatitis C, influenza A virus, avian influenza virus, and human immunodeficiency viruses.

### EXAMPLE 1

### Sorbent Synthesis

800 g (3.5 mol) of antimony trichloride (>99.5%) was dissolved into 400 mL of concentrated (370%) hydrochloric acid solution and then diluted with 1 L of 1 M HCl. The acidic solution was cooled on an ice-bath to 15±5° C. and, under vigorous stirring, about 220 mL of a 50% H₂O₂ solution (˜3.9 mol) was added in drop-wise fashion, so that reaction temperature did not exceed 50° C. (the reaction is exothermic). The oxytrichloride (SbOCl₃) solution formed was then mixed with 4 L of aqueous 25% monobasic sodium phosphate and 780 mL of aqueous 10% sodium metasilicate pentahydrate. This produced a white gel-like product, the “primary gel”, after being left at room temperature overnight, which was subsequently washed with 5 L of an aqueous 5% NaCl solution and then 5 L of deionized water. The washed “primary gel” was treated further with 2 L of a 5% sodium chloride (NaCl) solution and then with 2 L of a 5% sodium bicarbonate (Na₂CO₃) solution until a pH between 8 and 9 was achieved giving a slightly basic “primary gel.” The slightly basic “primary gel” was centrifuged at room temperature at 3000 rpm (2,054×g) for 12 minutes. The supernatant was discarded and the resulting white, powdery pellet was dried in a vacuum oven at 80-100° C. and 15 mm Hg for 12 hours. Once dried, the pellet was ground to a powder using a mortar and pestle, transferred to 4 L Erlenmeyer flask, and activated by careful addition of concentrated nitric acid (HNO₃, 70%, 1:1 w/v, giving off nitrogen dioxide) and kept for 2 hours at room temperature. The final product was washed several times with deionized water by repeated centrifugations as described above, until the pH of the filtrate measured between 4.8 and 5.5. The residue (the final product) was then collected, more solvent was added, centrifuged as above, and dried in a vacuum oven at 90° C. and 15 mmHg. The aforementioned sorbent copolymer was synthesized three separate times, using the synthetic method above. Yields of collected white solid product varied between 1200 and 1300 g.

### EXAMPLE 2

### Standard Testing Procedures

To (the) 100 mL of either 0.1 M NaCl or 0.1 M CaCl₂ (aqueous solutions) was added 1 g of the sorbent copolymer, and the mixture was shaken for 24 hours. The mixture was then centrifuged, the supernatant was filtered through a 0.45 μm filter, and cation concentration was determined by titration method, calorimetric analysis or mass-spectrometry. Sorption capacity was calculated according following equation (Ai−A)·V/M, where Ai is the initial cation concentration (Eq/L), A is the cation concentration after contact with the ion exchanger, V is the volume of solution and M is the mass of the ion exchange material.

Examples of the results achieved from testing batches of the aforementioned sorbent copolymer according to the standard testing procedures outlined above are shown in TABLE 2, below:

TABLE 2
Sorption capacity of copolymer

| | Na⁺ (mEq/g) | Ca²⁺ (mEq/g) |
|---|---|---|
| Sorbent Batch 1 | 3.40 | 4.25 |
| Sorbent Batch 2 | 3.45 | 4.20 |
| Sorbent Batch 3 | 3.35 | 4.30 |

As TABLE 2 shows, the variation in sorption capacity between batches does not exceed 0.03 mEq/g for Na⁺, and 0.02 mEq/g for Ca²⁺.

### EXAMPLE 3

### Treatment of Rabies in a Mouse Model

Rabies is an acute viral disease of mammals, most often transmitted via the bite of a rabid animal. Rabies virus infects the central nervous system, causing encephalopathy and, ultimately, death. Domestic and wild animals are susceptible to it, as are humans. Rabies is easily reproduced in white mice, and animals with an intracerebral infection develop encephalitis. In practice, a diagnosis of rabies is made on the basis of epizoological data, the patient's symptoms (e.g., fever, headache, general malaise, insomnia, anxiety, confusion, slight or partial paralysis, excitation, hallucinations, agitation, hypersalivation, difficulty swallowing, and hydrophobia), and the results of laboratory tests (including a bioprobe using white mice).

The copolymer sorbent of the present invention as a prophylactic was effective at preventing rabies in white mice. When the copolymer preparation was administered intravenously at 100-250 mg/kg and/or intramuscularly at 50 mg/kg of body weight, respectively, during the period 1 to 9 days after infection for the appearance of clinical signs, favorable results were obtained, as shown in TABLE 3 below.

TABLE 3
Survival rate of rabies-infected mice after copolymer administration (250 mg/kg)

| | | | | Time to |
|---|---|---|---|---|
| Copolymer | Copolymer Administration | Virus | Percent | Death |
| Administration Time | Route | Dilution | Survival | (days) |
| Not administered | untreated control | unit dose | 0 | 7.3 |
| | | 10⁻¹ | 50.0 | 7.2 |
| Prophylactic (before | intramuscular | unit dose | 68.0 | 7.75 |
| virus infection) | | 10⁻¹ | 84.0 | 5.5 |
| | intravenous | unit dose | 52.0 | 8.0 |
| | | 10⁻¹ | 76.0 | 8.66 |
| | Oral | unit dose | 43.0 | 7.4 |
| | | 10⁻¹ | 90.0 | 7.0 |
| | intramuscular and | unit dose | 43.5 | 9.4 |
| | intravenous every other day | 10⁻¹ | 76.0 | 8.0 |
| | intramuscular and | unit dose | 34.0 | 8.75 |
| | intravenous every other day | 10⁻¹ | 84.0 | 9.0 |
| | oral and intravenous every | unit dose | 51.5 | 8.4 |
| | other day | 10⁻¹ | 84.0 | 12.5 |
| On appearance of clinical | intramuscular | unit dose | 20.0 | 7.8 |
| symptoms, 6 days after | | 10⁻¹ | 80.0 | 7.5 |
| infection | intravenous | unit dose | 67.0 | 8.5 |
| | | 10⁻¹ | 90.0 | 12.0 |
| | oral | unit dose | 63.0 | 10.0 |
| | | 10⁻¹ | 90.0 | 12.0 |
| | oral and intramuscular | unit dose | 10.0 | 7.0 |
| | every other day | 10⁻¹ | 100.0 | 0 |
| | intravenous and | unit dose | 70.0 | 10.0 |
| | intramuscular every other day | 10⁻¹ | 100.0 | 0 |
| | Oral and intravenous | unit dose | 40.0 | 9.0 |
| | every other day | 10⁻¹ | 100.0 | 0 |
| On appearance of clinical | intravenous | unit dose | 70.0 | 9.0 |
| symptoms, 7 days after | | 10⁻¹ | 100.0 | 0 |
| infection | oral | unit dose | 30.0 | 10.5 |
| | | 10⁻¹ | 60.0 | 10.5 |
| | oral and intramuscular | unit dose | 40.0 | 9.0 |
| | every other day | 10⁻¹ | 90.0 | 12.0 |
| | intravenous and | unit dose | 90.0 | 9.0 |
| | intramuscular every other | 10⁻¹ | 90.0 | 9.0 |
| | day | | | |
| | oral and intravenous | unit dose | 60.0 | 9.85 |
| | every other day | 10⁻¹ | 100.0 | 0 |

The survival rate in rabies-infected mice given the sorbent copolymer of the present invention was 90%, and resolution of clinical symptoms was observed in the remaining mice.

### EXAMPLE 4

### Aujeszky's Disease

Aujeszky's disease—pseudorabies, infectious bulbar palsy, mad itch, frenzied scratching—is an acute disease of agricultural animals of all species, fur-bearing animals, and rodents. It is characterized by signs of infection of the brain, intense itching, and scratching (in all animals, except swine).

As shown in TABLE 4, below, the combined administration of the sorbent copolymer of the present invention—orally and intramuscularly—for prophylaxis or upon the appearance of clinical signs of the disease, prevented the proliferation of the virus in the organism of the test animals, as demonstrated by immunofluorescent analysis (IFA).

TABLE 4
Suppression of Aujeszky's disease virus replication in rabbits
by 30% sorbent copolymer suspension

| | | Activity of a 30% | |
|---|---|---|---|
| Animal ID | | suspension in | Titer of serum |
| No. | Autopsy result | organs, by IFA | antibodies, by IFA |
| | Aujeszky's disease virus used for | 1:1500 | |
| | infecting rabbits | | |
| 1 | Lung with hemorrhaging, liver normal | 1:100 | 1:45 |
| 2 | Lung with point hemorrhaging, liver | 1:200 | 1:135 |
| | normal | | |
| 3 | Lung and liver normal | neg | neg |
| 4 | Lung and liver normal | n/t | neg |
| 7 | Lung and liver normal | n/t | 1:45-135 |
| 9 | Lung dark red with hemorrhaging, | 1:100 | n/t (died) |
| | liver enlarged | | |
| 11 | Lung dark-cherry color with | 1:50 | neg |
| | hemorrhaging, liver enlarged | | |
| 12 | Lung dark-cherry color with | n/t | 1:135 |
| | hemorrhaging, liver enlarged | | |
| 15 | Lung dark red with hemorrhaging, | 1:100 | n/t (died) |
| | liver very enlarged | | |
| 20 | Lung and liver normal | n/t | neg |
| 21 | Lung pink, dark red hemorrhaging in | n/t | neg |
| | some places, liver normal | | |
| 22 | Lung pink, dark red hemorrhaging in | 1:100 | 1:45-135 |
| | some places, liver normal | | |
| 23 | Lung pink, dark red hemorrhaging in | n/t | 1:135-405 |
| | some places, liver normal | | |
| 25 | Lung with hemorrhaging, liver normal | 1:50 | n/t (died) |
| 26 | Lung and liver normal | 1:50 | n/t (died) |
| 28 | Lung dark-cherry color, entirely | n/t | neg |
| | affected, liver normal | | |
| 29 | Lung pale pink, liver pale | 1:200-400 | 1:45-135 |
| 31 | Lung pale pink, in places pale blue | 1:50 | neg |
| | with point hemorrhages, liver normal | | |
| 34 | Lung and liver normal | n/t | neg |
| 37 | Lung and liver normal | 1:100 | neg |

### EXAMPLE 5

### Rotavirus Infection

Rotavirus infection of cattle (diarrhea of newborn calves) is an acute, contagious disease of newborn calves characterized by infection of the gastrointestinal tract. The disease is widespread in all geographical regions of the world. It is also the most common cause of severe diarrhea among human children, and leads to the hospitalization of roughly 55,000 children in the United States each year. Rotavirus causes the death of more than 600,000 children each year, worldwide.

The sorbent copolymer of the present invention was tested against rotavirus infection in cattle. Rotavirus infection manifests itself clinically only in calves in the form of recurring diarrhea. Morbidity approaches 90%, and mortality approaches 20-50%. The disease is observed most frequently in late winter and early spring. Calves usually become infected in the first hours after birth. The agent of rotavirus infection in cattle is assigned to the family Reoviridae, genus Rotavirus. Rotavirus virions are spherical particles 65-75 nm in diameter, comprising an inner core and an inner and an outer capsid.

Intramuscular administration of the sorbent copolymer of the present invention at a rate of 10 mg/kg of live weight and oral administration at a rate of 40 mg/kg protected calves from death. No virus was detected in stool samples after the preparation was administered. The results are presented in TABLE 5.

TABLE 5
Effect of sorbent copolymer on rotavirus infection and survival in calves

| | | | | Stool sample test results pre- or post- | | | |
|---|---|---|---|---|---|---|---|
| | | | Amount | administration | | | |
| Calf | Age | Route of | administered | via electron microscopy | | Via IFA | |
| No. | (days) | administration | (mg/kg) | pre | post | pre | post |
| 1 | 2-3 | p.o. | 40 | n/t | death | neg. | death |
| 2 | 3 | i.m. | 10 | >10⁸ rotavirus | no rotavirus | 1:20 | neg. |
| | | | | particles per | particles | | |
| | | | | ml detected | detected | | |
| 3 | 2 | p.o. | 40 | n/t | n/t | 1:10 | neg. |
| | | | | | | to | |
| | | | | | | 1:20 | |
| 4 | 3 | p.o. | 40 | >10⁸ rotavirus | death | 1:40 | death |
| | | | | particles per | | | |
| | | | | ml detected | | | |

n/t = “not tested”

40 mg/kg of the sorbent copolymer administered orally was not sufficient to protect calf number four, which had a high concentration of the rotavirus in the stool (1:40 in IFA).

### EXAMPLE 6

### The Sorbent Copolymer was Tested Against Bovine Leucosis Virus, in Cattle

Bovine leucosis virus is the causative agent of Enzootic Bovine Leucosis, a viral infection of lymphocytes, in cattle. Cattle may be infected with the virus, yet not show any outward clinical manifestations of infection; more than 80% of cows at some dairies have positive titers for the virus, and there is no known treatment. Bovine leucosis virus can cause significant economic loss through decreased milk production, and through tumors in the heart, abomasum, spinal cord, uterus, and lymph nodes.

Blood was drawn from 30 calves aged 4-6 months, and from 10 cows aged 3-5 years. The blood was screened via standard hematological methods, and was tested via immunodiffusion reaction (IDR), and the results are presented in TABLE 6. As shown therein, antibodies specific to bovine leucosis virus were detected in the sera of 11 animals. In three calves, the neutrophils showed a nucleus shift to the left, which the inventors define as a cellular sign of leucosis. Based on the serological and hematological test results, cows 40, 49, 84, and 1523 bore active bovine leucosis infections.

On the day that the sorbent copolymer was administered, but before its administration, additional blood samples were taken from animals with leucosis and from those suspected of having leucosis. This blood was tested serologically and hematologically, and the results are presented in TABLES 6, 7, and 8. Calves 6271, 7245, 8449, 8483 (average weight 100 kg) received 10 cm³ intravenously of a 12% suspension (in deionized water) of the sorbent copolymer; cows 40, 49, 482, 1523, 1664, and 1789 (average weight 200-300 kg) received intravenous injections at the rate of 15 cm³ of the 12% suspension.

One day after the first dose, the sorbent copolymer was administered again via a combination of intravenous and oral methods: each calf received 10 and 15 cm³ of a 12% suspension (in deionized water) of the sorbent copolymer (e.g., 10 cm³ i.v. and 15 cm³ oral, or a total of 4.2 g, which was approximately 42 mg/kg); and each cow received 15 and 30 cm³ of a 12% suspension (in deionized water) of the sorbent copolymer (e.g., 10 cm³ i.v. and 15 cm³ oral, or a total of 7.2 g, which was between 24 and 36 mg/kg).

TABLE 6
Hematologic and serologic test results for leucosis, in cows aged 3-5 years

| | | Animal Number | | | | | | | | | |
|---|---|---|---|---|---|---|---|---|---|---|---|
| Blood values (normal | Sampling | control | | | | sorbent copolymer | | | | | |
| variation) | day | 84 | 91 | 175 | 1855 | 40 | 49 | 482 | 1523 | 1664 | 1789 |
| Leucocytes | 0 | 24.6 | N | N | N | 19.0 | 28.6 | N | 264 | N | N |
| (4.5-12.0 thousand/μl) | 42 | 235 | n/t. | n/t | 126 | 23.6 | 151 | 12.4 | 272 | 112 | 6.2 |
| | 44 | n/t | n/t | n/t | n/t | 24.8 | 18.4 | 16.6 | 28.4 | 18.0 | 8.6 |
| | 51 | 29.0 | 8.8 | 10.4 | 10.4 | 318 | 20.8 | 134 | 203 | 11.8 | 6.2 |
| | 57 | n/t | n/t | n/t | n/t | 342 | 18.0 | 9.8 | 10.4 | 15.8 | 7.0 |
| Neutrophils, rod | 42 | 1.0 | | | 4.0 | 3.0 | | 1.0 | 1.0 | | |
| (2.0-5.0%) | 44 | | | | | 4.0 | 2.0 | | 1.0 | 1.0 | 4.0 |
| | 51 | | | | | | 1.0 | 2.0 | 2.0 | | |
| | 57 | | | | | | 1.0 | | | | |
| Neutrophils, segmented | 42 | 14.0 | | | 29.0 | 3.0 | 13.0 | 19.0 | 80 | | |
| (20.0-35.0%) | 44 | | | | | 8.0 | 7.0 | 14.0 | 11.0 | 220 | 19.0 |
| | 51 | 5.0 | | | | 9.0 | 170 | 170 | 7.0 | | |
| | 57 | | | | | | 11.0 | | | 18.0 | |
| Lymphocytes | 42 | 81.0 | | | 560 | 860 | 86.0 | 76.0 | 890 | | |
| (40.0-65.0%) | 44 | | | | | 870 | 89.0 | 79.0 | 85.0 | 66.0 | 68 0 |
| | 51 | 92.0 | | | | 91.0 | 81.0 | 70.0 | 91.0 | | |
| | 57 | | | | | 92.0 | 87.0 | | | 75.0 | |
| Eosinophils | 42 | 3.0 | | | 110 | 2.0 | | | | | |
| (3.0-8.0%) | 44 | | | | | 10 | 2.0 | 7.0 | 3.0 | 11.0 | |
| | 51 | 1.0 | | | | | | | | | |
| | 57 | | | | | | | | | 3.0 | |
| Basophils | 42 | | | | | | 10 | 4.0 | 2.0 | | |
| (0.0-2.0%) | 44 | | | | | | | | | | |
| | 51 | 2.0 | | | | | 1.0 | 11.0 | | | |
| | 57 | | | | | 2.0 | | | | 4.0 | |
| Monocytes | 42 | | | | | 1.0 | | | | | |
| (2.0-7.0%) | 44 | | | | | | | | | | 1.0 |
| | 51 | | | | | | | | | | |
| | 57 | | | | | | | | | | |
| Hemoglobin | 57 | | | | | 4.9 | 6.5 | 5.0 | 5.7 | 6.5 | 5.8 |
| (9.0-12.0 g %) | | | | | | | | | | | |
| Reactions | 0 | + | − | − | + | + | + | + | + | + | + |
| Immunodiffusion reaction | 42 | + | + | n/t | + | + | + | + | + | + | + |
| | 44 | + | n/t | n/t | n/t | + | + | + | + | + | + |
| | 51 | + | − | − | n/t | + | + | + | + | + | + |
| | 57 | + | n/t | n/t | n/t | + | + | + | + | + | + |

“n/t” = “not tested”;
“N” = “normal”

The results of TABLE 7 mirror those of TABLE 6, but the tests which provided the results of TABLE 7 were performed at a different testing facility for the sake of confirmation.

TABLE 7
Hematologic and serologic test results for leucosis, in cows aged 3-5 years

| | | Animal Number | | | | | |
|---|---|---|---|---|---|---|---|
| Blood values | Sampling | sorbent copolymer | | | | | |
| (normal variation) | day | 40 | 49 | 482 | 1523 | 1664 | 1789 |
| Leucocytes | 57 | 12.0 | 10.7 | 9.8 | 11.5 | 10.0 | 10.5 |
| (4.5-12.0 × 10³/ | | | | | | | |
| μl) | | | | | | | |
| Neutrophils, rod | 57 | none | 2.0 | 1.0 | 3.0 | 2.0 | 1.0 |
| (2.0-5.0%) | | | | | | | |
| Neutrophils, | 57 | 17.0 | 15.0 | 20.0 | 22.0 | 25.0 | 15.0 |
| segmented | | | | | | | |
| (20.0-35.0%) | | | | | | | |
| Lymphocytes | 57 | 80.0 | 78.0 | 76.0 | 70.0 | 68.0 | 82.0 |
| (40.0-65.0%) | | | | | | | |
| Eosinophils | 57 | 2.0 | 2.0 | none | 3.0 | 3.0 | none |
| (3.0-8.0%) | | | | | | | |
| Basophils | 57 | 1.0 | 1.0 | 2.0 | none | 1.0 | 1.0 |
| (0.0-2.0%) | | | | | | | |
| Monocytes | 57 | none | 2.0 | 1.0 | 2.0 | 1.0 | 1.0 |
| (2.0-7.0%) | | | | | | | |
| Erythrocytes | 57 | 4.3 | 4.5 | 5.0 | 5.7 | 6.1 | 4.0 |
| (5-7.5 × 10⁶/μl) | | | | | | | |
| Hemoglobin | 57 | 6.0 | 6.3 | 8.0 | 7.5 | 9.0 | 7.0 |
| (9.0-12.0 g %) | | | | | | | |
| ESR, mm/hr | 57 | 0.5 | 0.6 | 0.6 | 0.6 | 0.5 | 0.6 |

The results shown in TABLE 8 mirror those of TABLES 6 and 7, above, but the results in TABLE 8 are from calves instead of cows.

TABLE 8
Hematologic and serologic test results for leucosis, in calves aged 4-6 months

| | | | Hematological tests | | | | | |
|---|---|---|---|---|---|---|---|---|
| | | | | Neutrophils (%) | | | | |
| Animal | Sample | IDR | Leucocytes | Rod | Segmented | Eosinophils | Basophils | Lymphocytes |
| No. | day | Result | (4.5-12.0 × 10³/μl) | (2.0-5.0) | (20.0-35.0) | (3.0-8.0%) | (0.0-2.0%) | (40.0-65.0%) |
| 6271 | 0 | + | N | | 26.0 | | | 74.0 |
| | 40 | + | 7.8 | | | | | |
| | 42 | + | 9.2 | | | | | |
| | 49 | + | 12.8 | | | | | |
| 8449 | 0 | − | 13.4 | 2.0 | 42.0 | | | 56.0 |
| | 40 | − | 8.2 | | | | | |
| | 42 | − | 9.0 | | | | | |
| | 49 | − | 14.2 | | | | | |
| 7245 | 0 | + | 12.4 | | | | | |
| | 40 | + | 7.6 | | | | | |
| | 42 | + | 9.0 | | | | | |
| | 49 | + | 10.1 | | | | | |
| 8483 | 0 | − | 20.0 | 6.0 | 25.0 | 1.0 | | 68.0 |
| | 40 | − | 10.0 | | | | | |
| | 42 | − | 9.4 | | | | | |
| | 49 | − | 20.8 | | | | | |
| 8458 | 0 | + | N | 2.0 | 32.0 | 1.0 | | 65.0 |
| | 40 | + | 10.6 | | | | | |
| | 49 | + | 18.0 | | | | | |
| 6250 | 0 | − | N | 2.0 | 14.0 | | 1.0 | 81.0 |
| | 49 | − | 18.8 | | | | | |
| 6267 | 0 | − | N | | | | | |
| | 49 | − | | | | | | |
| 7251 | 0 | − | N | | | | | |
| 7198 | 0 | − | N | | | | | |
| 7244 | 0 | − | N | | | | | |
| 7194 | 0 | − | N | | | | | |
| 7240 | 0 | − | N | | | | | |
| 7201 | 0 | − | N | | | | | |
| 8395 | 0 | − | N | | | | | |
| 8454 | 0 | − | N | | | | | |
| 8471 | 0 | − | N | | | | | |
| 8469 | 0 | − | N | | | | | |
| 9233 | 0 | − | N | | | | | |
| 8465 | 0 | − | N | | | | | |
| 8450 | 0 | − | N | | | | | |
| 9220 | 0 | − | N | | | | | |
| 9223 | 0 | − | N | | | | | |
| 9218 | 0 | − | N | | | | | |
| 8437 | 0 | − | N | | | | | |
| 9226 | 0 | − | N | | | | | |
| 9224 | 0 | − | N | | | | | |

“N” = “normal”

### EXAMPLE 7

### Hepatitis C

Hepatitis C virus (HCV) subtype 1b (HCV-1b, or HCV type 1b) was isolated from the serum of a chronically infected HCV patient. The HCV-1b genotype is the most widespread in Russia and is also common in America, where about 70% of HCV-infected persons are infected with genotype 1. In the studies below, the infectious dose of HCV was equal to 10.0 TD₅₀ (the 50% cytotoxic dose).

Fifty white mice, each weighing about 18 grams, were assigned to five groups of ten mice each. Each mouse was given two 200 μL intravenous injections of sorbent copolymer, with 12 hours between the first and second administrations. Groups 1, 2, 3, 4, and 5 received 50, 100, 200, 400, and 800 mg/kg of sorbent copolymer in tridistilled water, respectively, with each intravenous injection. All animals were kept for observation for one month after the second and final administration, except Group 4. The injection protocol caused no apparent pathologies, and no animals died before the experimental enpoints.

One week after administration of sorbent copolymer, the Group 4 mice (which received 400 mg/kg with each injection) were injected intravenously with HCV-1b. The Group 4 mice were sacrificed one week after infection, and blood, serum, and brain tissue was collected. The brain tissue was homogenized in phosphate-buffered saline, and all samples were centrifuged for 15 minutes at 3,000 rpm and 4° C. The resulting supernatant was probed for HCV antigens via hemagglutination reaction (Clarke D H, Casals J. Am J Trop Med Hyg. 1958; 7(5):561-73), with the results shown in TABLE 9.

TABLE 9
Titer of HCV antigen determined by hemagglutination test

| | In blood | | | In brain | | |
|---|---|---|---|---|---|---|
| | Sorbent Copolymer | | | Sorbent Copolymer | | |
| | given | | | given | | |
| Mouse | After | Before | Without | After | Before | Without |
| Number | infection | infection | preparation | infection | infection | preparation |
| 1 | 1:20 | 1:256 | 1:80 | 1:1280 | 1:5120 | 1:640 |
| 2 | 1:80 | 1:128 | 1:320 | 1:1280 | 1:5120 | 1:320 |
| 3 | 1:40 | 1:256 | 1:80 | 1:2560 | 1:2560 | 1:640 |
| 4 | 1:80 | 1:128 | 1:80 | 1:1280 | 1:5120 | 1:320 |
| 5 | 1:80 | 1:128 | 1:80 | 1:1280 | 1:2560 | 1:640 |
| Average | 1:60 | 1:179 | 1:128 | 1:1536 | 1:4096 | 1:512 |

As the data of TABLE 9 show, sorbent copolymer administration was correlated with decreased HCV antigenic activity in serum and in brain supernatant. On average, serum HCV antigenic activity was decreased three fold in animals that received sorbent copolymer, while brain supernatant HCV antigenic activity was increased eight fold.

The supernatant from Group 4 mice was also tested for infectivity in porcine embryonic kidney cells. Porcine embryonic kidney cells were grown in 24-well plates, in Medium 199 supplemented with 10% fetal bovine serum, L-glutamine, and penicillin/streptomycin (100 μg/mL, each). One day post-passage, cells were exposed to mouse-tissue supernatants. A strain of HCV that is highly pathogenic to porcine embryonic kidney cells, obtained from the Ivanovsky Institute of Virology (“NARVAK”), Moscow, Russia, was used as a positive control. Either 6 or 7 days after infection, when maximal cytopathogenic action had developed, the titer was assayed using the method of Reed-Muench (Dulbecco R. 1998. End-point method-measurement of the infectious titer of a viral sample. p. 22-25. In: Dulbecco R. and Ginsberg H. S. (eds.), Virology, 2d ed. Lippincott Williams & Wilkins, Philadelphia, Pa.) and the results are displayed in TABLE 10.

TABLE 10
Infectious titer of HCV (log TD50)

| | In blood | | | In brain | | |
|---|---|---|---|---|---|---|
| | Sorbent Copolymer | | | Sorbent Copolymer | | |
| | given | | | given | | |
| Mouse | After | Before | Without | After | Before | Without |
| Number | infection | infection | preparation | infection | infection | preparation |
| 1 | 2.7 | 2.3 | 5.0 | 6.5 | 3.5 | 6.0 |
| 2 | 2.3 | 2.3 | 4.5 | 3.5 | 4.5 | 7.0 |
| 3 | 2.7 | 3.0 | 5.2 | 4.3 | 4.0 | 7.0 |
| 4 | 3.0 | 3.2 | 5.1 | 5.0 | 3.6 | 4.8 |
| 5 | 3.0 | 3.5 | 5.0 | 5.3 | 4.6 | 5.7 |
| Average | 2.7 | 2.8 | 5.0 | 4.9 | 4.0 | 6.1 |

TD₅₀ for the control HCV strain was 10.0.

As can be seen from TABLE 10, treatment with sorbent copolymer more than 1.5 log reduced infectious activity of HCV virus in blood of infected mice and on the average the titers were reduced by 1.2 log under the influence of a preparation in animal a brain.

The data of TABLES X and X demonstrate the preventive effect of the sorbent copolymer against HCV infection in mice. Although two doses of sorbent copolymer administered 1 week prior to HCV infection failed to decrease HCV antigenic activity in mouse serum and brain supernatant, administration of sorbent copolymer was correlated with reduced infectious activity of HCV in serum and brain supernatant by 2.3 log TD50 (average data). Increased HCV antigenic activity in brain supernatant from infected animals treated with the sorbent copolymer may be related to a reduction of cell sensitivity to the replication of mature virions, thus resulting in a population of newly-generated virus that is substantially enriched for defective non-infectious particles bearing antigenic activity.

### EXAMPLE 8

### Inactivating Activity of Sorbent Copolymer on Influenza Virus In Vivo

Annual influenza outbreaks are a global public health concern. According to the World Health Organization, between 3 and 5 million people worldwide get flu every year (VVWR 2002). Influenza infection aggravates chronic somatic diseases and causes the development of complications, sometimes leading to death. The “additional” death rate from flu may range from tens to hundreds per 105 of population, and during a flu epidemic this parameter may reach 1,000 cases per 10⁵ of population.

In recent years, a highly pathogenic flu strain—influenza A/H5N1—has circulated among wild and domestic birds, sometimes causing lethal infections in humans and increasing the likelihood that a pandemic (a worldwide epidemic) will develop. This demonstrates the need for effective flu remedies. In the United States the only effective anti-flu medication available is the neuraminidase inhibitor Tamiflu (Roche Pharmaceuticals), which is available in limited supply or has been subject to production shortages, and has been reported to cause dangerous psychological effects in some patients. Another anti-flu medication is Arbidol, an antiviral drug manufactured by Masterlek (Moscow, Russia). Its antiviral inhibitory effect is still being tested and the current results range from being well accepted in pharmaceutical industry to accepted but with a dose of suspicion. Arbidol (CAS No. 131707-23-8) has been tested mainly in Russia and China, where it has been shown to be effective against avian flu, suggesting it might be a more affordable and cost-effective drug than the more widely used Tamiflu. According to the manufacturer, Arbidol exhibits immunomodulation as well as a specific anti-influenza action against the influenza A and influenza B viruses. Arbidol prevents contact between the virus and host cells, and prevents penetration of virus particles into the cell by inhibiting the fusion of the virus lipid shell to cell membranes. Arbidol possesses interferon inducing action, elicited by stimulating the humoral reaction and the phagocytic function of macrophages. Because of their various shortcomings, in the event of a flu pandemic none of the currently available medications will suffice. Consequently, the development of effective anti-flu preparations is imperative.

The effects of sorbent copolymer administration on influenza type A infection were studied in mice. Solutions of sorbent copolymer at 20 mg/ml and 50 mg/ml were prepared using sterile distilled water. Suspension of the obtained preparation was maintained for 4 hours at 4° C., then centrifuged at 2000 rpm for 15 minutes and the supernatant was used in the experiment. Type A flu virus A/Aichi//2/68 (H₃N₂) adapted to lungs of mice at a dose of 100 LD 50 was used. Infectious activity of the virus was determined by the Reed-Muench accumulative titration method (Dulbecco R. 1998. End-point method-measurement of the infectious titer of a viral sample. p. 22-25. In: Dulbecco R. and Ginsberg H. S. (eds.), Virology, 2d ed. Lippincott Williams & Wilkins, Philadelphia, Pa.) of infecting mice by various cultivations of virus suspension.

Specificity of the virus infection was controlled by hemagglutination reaction and by the method of passage of virus suspension of lungs infected by a virus A/Aichi2/68 of mice, in chicken embryos. During experiments the titration of a virus was conducted with monospecific serum to exclude the possibility of contamination.

Inbred white mice weighing 17-20 g were infected intranasally with 0.05 ml flu virus (corresponding to 100 LD50) while under a light ether narcosis. Tests with mice were conducted according to standard laboratory protocols. Before tests, all animals were quarantined for to 10 days. At all times, animals received standard food and were kept at equilibrium conditions.

Flu-infected mice and non-infected control mice were given the sorbent copolymer (50 mg/mL IM and 20 mg/mL PO) or received normal saline as a control. The survival rate over 11 days between these groups of mice was then compared.

Antiviral action of the sorbent copolymer was assessed by its ability to suppress viral infectious activity, which was measured by RNA in a qualitative polymerase chain reaction (PCR) and by qualititative titration of mice lung cell culture suspension with Madin-Darby Canine Kidney epithelial cells (MDCK cells).

Determination of viral titer in suspensions of lung cells infected with flu virus was performed by a standard method in cultured MDCK cells. PCR was carried out with primers according to the instruction of “GENBANK EUROTHECH.”

Investigation of the therapeutic activity of the sorbent copolymer was conducted according to method recommendations on tests and assessment of antiviral actions of anti-flu preparations. The sorbent copolymer was administered orally 3 times per day at a dose volume of 25 μL of the preparation at 20 mg/mil, and intramuscularly once a day at 0.1 ml at the concentration of 50 mg/ml. Administration of sorbent copolymer commenced 48 hours after the mice were infected intranasally, and continued for three days.

To compare relative efficacies, Arbidol was used at the recommended dose for 3 consecutive days. The control group of animals was dosed with a physiological solution (0.9% saline solution) under the same conditions.

To study the antiviral activity of the sorbent copolymer, a viral dose causing 70% lethal outcome of mice, was used. The infecting dose of a virus and tested concentration of a preparation was tested in at least 10 mice. Results of experiments were summarized and subjected to statistical analysis. Animals were observed within 21 days after the infection, taking into account that flu virus caused death of mice in treated groups as well as in the control group (mice not treated with any drugs).

Experimental results were analyzed by the index of survival rate in the group of treated animals compared to that of the control group, an index of protection and average life expectancy of mice. Data were subjected to statistical analysis, allowing an objective assessment of the quantitative results of life expectancy of tested and control animals, computed by medical-statistical model corresponding to Poisson distribution with correction of C. Guard (Mainell and E. Mainell, 1970).

Mice were sorted into four groups of 10 animals each: 3 test groups and 1 control group (the control of an infecting dose). Group I mice were infected but not treated with drugs (only with physiological saline). Group 2 mice were infected and were treated with sorbent at 50 mg/mL (100 μL IM). Group 3 mice were infected and then treated with 20 mg/ml (25 μL PO). Group 4 mice were infected and were treated with Arbidol. After infection, animals were maintained for 48 hours without treatment. After 48 hours had elapsed, animals receiving sorbent PO were treated 3 times per day for three consecutive days; animals receiving sorbent IM were treated once each day for three consecutive days.

Survival Rate of Animals

In the group which was infected with virus and did not receive the sorbent copolymer, death was first observed by the fifth day after infection; by day seven, 70% of mice had died. The relative life expectancy in a control group was 8.5 days.

As shown in TABLE 11, the sorbent copolymer produced a protective effect in mice experimentally infected with flu virus. The results show that the survival rate of flu-infected mice was 100% when those mice received sorbent copolymer at 50 mg/ml (100 μL IM, once per day). Relative average life expectancy of mice, in comparison with the control group (treated only with physiological saline), has increased to 42 days. At a sorbent copolymer dose of 50 mg/ml (100 μL IM, once per day) the index of protection was 100%. At a sorbent copolymer dose of 20 mg/ml (25 μL PO, three times per day), the index of protection was 42.9% and average life expectancy increased to 12.5 days.

At the Arbidol dosing regimen used, the survival rate of infected mice was 40%. Relative average life expectancy of mice given Arbidol, in comparison with the control group, increased to 12.5 days and the index of protection of Arbidol was 42.9%.

Infectious Activity.

TABLE 12 demonstrates the infectious activity of flu virus in mice after application of the preparation. Particularly the data obtained via titration of suspension of lungs infected by a flu virus in cell cultures MDCK. The results show that the infectious activity of a type A flu virus in lungs of mice was completely suppressed when those mice received sorbent copolymer once a day, intramuscularly, at 50 mg/ml. Concentration of a preparation at 20 mg/ml reduced viral infectious titer on average more than 2 times compared to a control group of mice which wasn't treated with a preparation.

Determination of Viral RNA by PCR

Flu virus RNA was not detected in animals treated once daily with sorbent copolymer, given intramuscularly at 50 mg/ml. Viral RNA was detected in all mice of the control group. In the groups where sorbent copolymer was not administered, flu virus RNA was found in 80% of animals that received 20 mg/ml sorbent copolymer orally three times per day. The PCR data obtained from mice treated with sorbent copolymer at 20 mg/ml fully coincide with data of viral infectious activity and with results on survival rate of mice.

In conclusion, this study of the antiviral activity of sorbent copolymer in mice experimentally infected with type A flu virus A/Aichi//2/68 (H₃N₂) showed that: 1) the sorbent copolymer given intramuscularly once per day at 50 mg/ml completely suppressed flu infection in mice, which was confirmed by the viral RNA PCR results and the 100% survival rate despite receiving a viral dose that caused death in 70% of controls; 2) at 20 mg/ml, the sorbent copolymer also demonstrated antiviral activity in mice infected by type A flu virus, producing a two-fold decrease of infectious titers of virus, and 50% increase in life expectancy of treated mice; and 3) the sorbent copolymer of the present invention given orally three times per day at 20 mg/ml possesses antiviral activity equal to that of Arbidol, but when given intramuscularly once per day at 50 mg/ml the antiviral activity of the sorbent copolymer substantially surpasses that of Arbidol.

TABLE 11
Antiviral activity of sorbent copolymer vs. type A flu virus A/Aichi/2/68 (H3N2) in mice

| | | | | | | | | Avg. life | | |
|---|---|---|---|---|---|---|---|---|---|---|
| | | | | | | | | expectancy | | |
| | Days post-infection | | | | | | | (days) | | Defense |
| | (number of mice died) | | | | | Total | Lethality | | Versus | Index |
| Antiviral preparation | 5 | 6 | 7 | 8 | 9 | deaths | (%) | Appr. | control | (%) |
| 50 mg/ml | — | — | — | — | — | 0 | 0 | 42.4 | 33.9 | 100 |
| QD, i.m. | | | | | | | | | | |
| 20 mg/ml | — | (1) | (1) | (2) | — | 4 | 40 | 20 | ÷11.5 | 42.9 |
| TID, per os | | | | | | | | | | |
| Arbidol | — | — | — | (2) | (2) | 4 | 40 | 20 | ÷11.5 | 42.9 |
| None | (3) | (3) | (1) | — | — | 7 | 70 | 8.5 | 0 | 0 |

Note:
The life expectancy was determined according medical statistic model, corresponding to Poisson distribution with correction S. Gurd (G. Mainell and E Mainell).

TABLE 12
Infectious activity of lung virus in mice after
administration of sorbent copolymer

| | | Markers of flu virus infectivity | |
|---|---|---|---|
| Dose of preparation | Mouse ID | By PCR | MDCK cell culture titer log |
| 50 mg/kg | 1 | Neg | 0 |
| | 2 | Neg | 0 |
| | 3 | Neg | 0 |
| | 4 | Neg | 0 |
| | 5 | Neg | 0 |
| Average in group | | Neg | 0 |
| 20 mg/kg | 1 | + | 2 |
| | 2 | + | 4 |
| | 3 | + | 3 |
| | 4 | + | 2 |
| | 5 | − | 0 |
| Average in group | | + | 2.2 |
| Without preparation | 1 | + | 5 |
| | 2 | + | 5 |
| | 3 | + | 6 |
| | 4 | + | 5 |
| | 5 | + | 4 |
| Average in group | | + | 5 |

### EXAMPLE 9

### Inactivating Activity of Sorbent Copolymer on Influenza A Virus Subtype H5N1 (“Bird Flu”)

Although avian influenza A viruses usually do not infect humans, since 1996 a number of Asian and Middle Eastern countries have reported confirmed instances of avian influenza A virus infections (“bird flu” or “avian influenza”) in humans. Most of these cases have occurred in people having direct or close contact with H5N1-infected poultry or with H5N1-infected surfaces. However, research into the genes comprising the “Spanish Flu”—which killed between 20 and 40 million people worldwide between 1918 and 1919—shows that those genes are adapted to both birds and humans and suggests that the Spanish Flu originated in birds.

The possibility of spread to humans can be limited somewhat by destroying infected domestic fowl, but not stopped. Despite treatment, 70% of people infected with bird flu died. In July 2005, influenza A H5N1 was detected in Russia and in many regions of Russian Federation: in Western Siberia, Ural and Astrakhan areas.

Avian influenza can be lethal to humans, but people do not transmit it to one other. However, people may become infected from infected birds. Furthermore, some animals (e.g., pigs) easily catch human and avian influenzas. When epizooty of the bird's flu coincides with a human flu epidemic (and they usually fall to the same months), viruses of both types can be found in pigs.

Simultaneous propagation of human and avian influenza viruses in pigs can lead to re-assortment and evolution of a new “hybrid” virus in which the “bird” proteins—antigens of type A bird flu virus—will be combined with the ability to be transmitted to humans. Then, disaster looms: the new pathogen would be infectious to humans and also lethal. Thus there is a real threat of appearance of new pandemic strain.

As forecast by World Health Organization, in coming years there is a possibility of occurrence of a new type bird flu virus, to which people will not have an immunity, which can lead to development of pandemic.

There are no vaccines against avian influenza, and the existing anti-flu medicines are either ineffective or not sufficiently available. Consequently, there is a need for the creation of new means of treatment and prophylaxis against the flu.

Preliminary preclinical studies on the antiviral activity of the sorbent copolymer of the present invention in pneumonic mice infected with influenza A H5N3, a close variant of pathogenic type H5N1, were conducted. The study showed that in certain conditions the sorbent copolymer may protect animals from infection caused by a type A bird flu virus. Treatment with the sorbent copolymer led to increased life expectancy of the infected animals, and in certain cases the survival rate was as high as 70%. Thus, it is expected that the sorbent copolymer is useful for treatment of avian influenza.

In the following studies, a highly pathogenic influenza A H5N1 was used, isolated during the outbreak among domestic birds in Novosibirsk Region in July 2005. The strain is actively reproduced in cultured porcine embryonic kidney cells (PEKCs), which were used for studying the infectious properties of the virus. Nutrient media used in the work included: serum, antibiotics, and sterile, disposable culture dishes for the cultivation of virus.

The sorbent copolymer was in the form of a fine powder. Suspensions of the sorbent copolymer were prepared using distilled water or a physiological solution (0.9% saline), and sedimentation of the sorbent copolymer from suspension was carried out by centrifugation for 10-15 min at 4° C., which pelleted any heavy particles while leaving smaller particles in the supernatant. The supernatant so obtained was used for studying its antiviral activity against flu virus H5N1. In other experiments, a longer natural sedimentation was conducted, and that supernatant was used for the detection of antiviral effect.

The results obtained are presented in TABLES 13-15.

Sorbent copolymer suspensions of different concentrations were overlaid upon the cells in vitro. TABLE 13 shows that the sorbent copolymer suspended in a physiological solution or in distilled water, at different concentrations, is capable of actively suppressing infectious activity of bird flu virus H5N1. Complete suppression of viral infectious activity was observed after the addition of sorbent copolymer suspension at a concentration of 3.12 mg/mL, with exposure for 1 hour.

Sorbent copolymer suspension at 1.5 mg/mL was able to suppress infectious activity of type A bird flu virus by more than 10,000 fold when the exposure lasted either 30 minutes or 1 hour. In contrast, the physiological solution without sorbent copolymer added did not produce any detectable antiviral activity.

It was of interest to determine whether the physiological solution would retain antiviral activity after complete sedimentation of the sorbent copolymer. For that purpose, a physiological solution containing 0.3 mg/ml sorbent copolymer was used. After overnight natural sedimentation, the supernatant was collected and added to viral media and incubated for 1 hour, followed by detection of residual infectious activity. TABLE 14 demonstrates that the infectious activity after treatment with sorbent copolymer was less than 1.01 log, while infectious activity after treatment with control solution was 7.01 log TCID 50/ml.

In another experiment, a suspension of sorbent copolymer was pelleted by centrifugation at 2,000 rpm for 5 minutes. Material containing bird flu virus was added to the supernatant obtained. It was shown that cleared supernatant after centrifugation at 2,000 and 16,000 rpm still possessed high H5N1 antiviral activities. Virtually a complete inactivation of virus occurred at significantly high titers of virus in the control—6.01 log TCID50/ml.

It was also of interest to determine whether a pellet of sorbent copolymer recovered after centrifugation possessed antiviral activity. TABLE 14 shows that a re-suspended pellet of sorbent copolymer in a small volume of physiological solution is also capable of completely suppressing the infectious activity of bird flu virus within 1 hour.

Table 3 shows that supernatant obtained after centrifugation of sorbent preparation preserved a high anti viral properties and when diluted 2 times, and in case of 4 fold dilution it decreased anti viral activity almost by 100,000 fold compared to the controls.

However, the antiviral properties of supernatant decline after storage at 4° C. for 5 days. Studies showed that under those conditions the sorbent copolymer solution was capable of suppressing infectious activity of type A bird flu virus by 1000 fold (see TABLE 15).

We then determined what concentration of sorbent copolymer is necessary to obtain a solution with high antiviral properties, after the centrifugal sedimentation of sorbent copolymer. As shown in TABLE 16, the antiviral properties of physiological solution after sedimentation of a 3.0 mg/mol sorbent copolymer solution were preserved. The supernatant produced complete inactivation of the infectious properties of type A bird flu virus (H5N1) after 10 minutes of supernatant application to the H5N1 viral material. Infectious titer which was 4.0 TCID 50/20 μL. Lower concentrations of sorbent copolymer in physiological solution did not suppress antiviral activities.

Next, we determined how long the supernatant, obtained after sedimentation of sorbent copolymer, exhibits antiviral activity against type A bird flu virus H5N1. TABLE 18 shows that the supernatant preserved maximal antiviral activity against type A bird flu virus for as long as 4 hours. However after 4 days the antiviral properties decreased and the supernatant was capable of reducing infectious activity of the virus by 1,000 times. After 8 days of storage of supernatant liquid at 4° C. the titers of virus of flu A after 30 minutes of exposure in supernatant decreased by 100 fold. Obtained data demonstrate the decrease of antiviral properties of supernatant containing sorbent copolymer after being stored at 4° C.

In conclusion, we have shown that: 1) the suspension of sorbent copolymer possesses high antiviral activity against a highly pathogenic type A bird flu virus (H5N1); 2) the high antiviral activity is demonstrated at different concentrations of the preparation in suspension up to 3 mg/mL; 3) the supernatant obtained after centrifugation at 2,000 and up to 16,000 rpm retains high antiviral capacity; 4) the ability to suppress infectious activity of virus is preserved in the solution obtained after sedimentation and 2-4 fold dilution of sorbent copolymer; 5) the data show that a pellet obtained following centrifugation of sorbent copolymer containing infectious bird flu virus, didn't show H5N1 infectious activity; and 6) different concentrations of sorbent copolymer suspensions, as well as the supernatant obtained after the centrifugal sedimentation of said suspensions, were not cytotoxic. Cell cultures treated with suspension of the preparation preserved a high level of viability and proliferative activity.

Antiviral properties of sorbent copolymer solution obtained after the centrifugal sedimentation of sorbent copolymer at 3.0 mg/ml facilitated a complete inactivation of infectious properties of type A bird flu virus (H5N1) within 10 minutes after the application of supernatant on virus containing material, the infectious titer of which reached 4.0 log TCID 50/20 μL. Lower concentrations of sorbent copolymer in the physiological solution did not suppress the infectious properties of A bird flu virus.

Furthermore, we have shown that water passed through a cartridge containing sorbent copolymer possesses measurable antiviral activity against type A bird flu virus. The data obtained indicate the decrease of antiviral properties of supernatant, containing sorbent copolymer, when stored at 4° C.

TABLE 13
Virulence action of sorbent suspension in different concentrations
corresponding to high pathogenic version of flu virus H5N1
Infectious titer of flu virus H5N1 for cells culture PEKC
(in 1 g TCID 50/ml)

| | Concentration of product (mg/ml) | | | | | | Control Of |
|---|---|---|---|---|---|---|---|
| | 50 | 25 | 12.5 | 6.25 | 3.12 | 1.5 | virus |
| Virus+suspension of | 0 | 0 | 0 | .0 | <1.0 | 2.5 | 6.7 |
| product - 30 min | | | | | | | |
| Virus+suspension of | 0 | 0 | 0 | 0 | 0 | 2.0 | 6.7 |
| product - 1 hour | | | | | | | |

TABLE 14
The ability of solutions after sedimentation of sorbent (0.3 mg/ml) to inactivate
the infectious properties of high pathogenic version of virus H5N1

| | Log TCID | | | Log TCID |
|---|---|---|---|---|
| Variants of tests | 50/ml | Variants of tests | | 50/ml |
| Supertanant after natural | <1.0 | Suspension of sorbent prior to | | 1.2 |
| sedimentation | | centrifuging | | |
| Sediment | 0 | Supernatant after | 2 000 rot/m | 0 |
| | | centrifuging | 5 min | |
| | | | 16 000 rot/m | 0 |
| | | | 10 min | |

| | | | |
|---|---|---|---|
| Without product | 7.0 | Physiological solution without product | 6.0 |

TABLE 15
The ability of different mixtures of supernatant after sedimentation of
product sorbent to decrease infectious activity of flu virus H5N1
during 30 minutes and 1 hour (the beginning concentration
of the product 3.0 mg/ml)

| | Infectious titer of flu virus H5N1 | | |
|---|---|---|---|
| Mixtures of | for cells culture PEKC | | |
| supernatant | (in log TCID 50/ml) | | |
| liquid | 30 min | 60 min | 4 days |
| Whole solution | 0 | 0 | 5.0 |
| 1:2 | 0 | 0 | [symbol] |
| 1:4 | 4.8 | 3.2 | [symbol] |
| 1:8 | 7.0 | 5.0 | [symbol] |
| Control of virus | 8.0 | 8.0 | 8.0 |

TABLE 16
Virulent action of supernatant liquid received after sedimentation of
sorbent on virus of bird's flu A H4N1 (in every 10 min. after processing

| Titer of virus | Virulent action of supernatant liquid received after | | | | | |
|---|---|---|---|---|---|---|
| of bird flu for | sedimentation of sorbent in concentration (mg/ml) | | | | | |
| cells cultures PEKC | 30.0 | 3.0 | 0.3 | 0.3:2 | 0.3:4 | Control of virus |
| In log TCID 50/ml | 0 | 0 | 3.0 | 4.0 | 3.8 | 4.0 |

TABLE 17
Virulent action of water passing through cartridge containing sorbent, on
virus of bird flu H5N1

| | Virulent properties of water tests | | | | | | | |
|---|---|---|---|---|---|---|---|---|
| | selected during passing through the cartridge | | | | | | | |
| Titer of virus of | filled with sorbent - number of water tests | | | | | | | |
| bird's flu for cells | processing by virus A of bird flu | | | | | | | |
| cultures PEKC | 1 | 2 | 3 | 4 | 5 | 6 | 7 | Control of virus |
| In log TCID | 5.5 | 4.2 | 4.8 | 4.8 | 4.0 | 3.5 | 5.5 | 5.5 |
| 50/ml | | | | | | | | |

TABLE 18
Dynamic of preserving of virulent properties of supernatant receiving
after sedimentation of product sorbent concerning to virus
A of bird flu (H5N1)

| | Titer of virus A of bird's flu (H5N1) after 30 min | | | |
|---|---|---|---|---|
| Titer of virus of | of exposition of supernatant liquid received after | | | |
| bird's flu for cells | sedimentation of product sorbent | | | |
| cultures PEKC in | (during of receiving of supernatant solution) | | | |
| log TCID 50/ml | 4 hours | 4 days | 8 days | Control of virus |
| Supernatant after | 0 | 4.1 | 5.0 | 7.0 |
| sedimentation at | | | | |
| 2000 rot/min | | | | |
| Supernatant after | 0 | 6.0 | 7.0 | 9.0 |
| sedimentation at | | | | |
| 16000 rot/min | | | | |

### EXAMPLE 10

### Activity of Sorbent Copolymer Against Human Immunodeficiency Virus

Testing the efficacy of the sorbent copolymer against human immunodeficiency virus (HIV) was conducted in two phases. First, the sorbent copolymer was added to cultures of cells used for cultivation of HIV, to determine whether the sorbent copolymer was cytotoxic. Second, the sorbent copolymer was tested in those cultures to determine its effect on HIV infection.

The human MT-4 T-cells line and laboratory strain HIV-1VS was used. This cell line was chosen because it is highly sensitive to HIV-1 and can be used for testing of antiviral preparations (Pauwels R., De Clercq E. Et. Al. Sensitive and rapid assay on MT-4 cells for direction of antiviral compounds against the AIDS virus. J Virol. Meth. 1987; 16:171-185).

A 50 mg/ml working solution of sorbent copolymer in distilled water was prepared, stored for 3 hours at 4° C., centrifuged at 2,000 rpm for 15 min, and then used to prepare working dilutions. Fresh working solutions were prepared for each experiment. Alternatively, a 50 mg/ml working solution of sorbent copolymer in distilled water was prepared, kept at room temperature, and then used to prepare working dilutions. Fresh working solutions were prepared for each experiment.

To assess cytotoxicity of the sorbent copolymer preparations, 400 μl of a cell suspension containing 2.5-3.0×10⁵ MT-4 cells/ml was added to 48-well cell culture plates. Then, 100 μl of a sorbent copolymer working dilution was added. Three wells were used for each dilution, and the plates were incubated for 3 to 4 days at 37° C. in a 3.5% CO₂ atmosphere.

After incubation for 3 to 4 days, live cells were counted in a hemocytometer in the presence of Trypan blue. Tests for each sample were performed in triplicates. For each working dilution concentration, in 3 experiments, the mean value of cell viability was determined. Cell viability in untreated controls was taken as 100%.

In TABLE 19, the working solution was prepared, stored for 3 hours at 4° C., centrifuged at 2,000 rpm for 15 min, and then used to prepare working dilutions. In TABLE 20, the working solution was prepared, kept at room temperature, and then used to prepare working dilutions. The values presented in TABLES 19 and 20 are the mean values from experiments performed in triplicate.

TABLE 19
Cytotoxic activity of sorbent copolymer

| Dilution | Live cells (absolute) | Live cells (relative, %) |
|---|---|---|
| H₂O control | 855470 | 100 |
| 1/5 | 588830 | 68.8 |
| 1/10 | 688820 | 80.52 |
| 1/100 | 933240 | 109.1 |
| 1/1000 | 733260 | 91.56 |
| 1/10000 | 783255 | 85.7 |
| 1/100000 | 866580 | 101.3 |

TABLE 19 clearly shows that that 1:10 dilution of the preparation can be used in further experiments.

TABLE 20
Cytotoxic activity of sorbent copolymer

| Dilution | Live cells (absolute) | Live cells (relative, %) |
|---|---|---|
| H₂O control | 940000 | 100 |
| 1/5 | 367200 | 39 |
| 1/10 | 512000 | 54.5 |
| 1/100 | 677520 | 72 |
| 1/1000 | 865211 | 92 |
| 1/10000 | 921340 | 98 |
| 1/100000 | 945121 | 100 |

TABLE 20 clearly shows that that 1:100 dilution of the preparation can be used in further experiments.

Culture medium containing HIV was mixed with sorbent copolymer working solution 1:1, at room temperature. After predetermined incubation intervals, samples were aliquoted and viral titer was determined by the final dilutions method. The TD50 was determined according to the method of Reed and Manch. Five-fold viral dilutions were prepared for each data point, and each dilution was tested in triplicate.

To assess anti-HIV activity of the sorbent copolymer preparations, 400 μl of a cell suspension containing 3.0-3.5×10⁵ MT-4 cells/ml was added to each well of a 48-well cell culture plate. Virus (100 μL from each dilution) and sorbent copolymer (working suspension) were added simultaneously. Cells were incubated for 7 days at 37° C. in a 3.5% CO₂ atmosphere, after which cytopathology was assessed. The data presented in TABLES 21, 22, and 23 are from experiments performed in triplicate. In TABLES 21 and 22, the working solution was prepared, stored for 3 hours at 4° C., centrifuged at 2,000 rpm for 15 min, and then used to prepare working dilutions. In TABLE 23, the working solution was prepared, kept at room temperature, and then used to prepare working dilutions.

TABLE 21

| Incubation time (min) | Titer (TD₅₀) |
|---|---|
| 3 | 10 (5.2) |
| 10 | 10 (5) |
| 30 | 10 (3.8) |
| 30* | 10 (5) |

*virus in the absence of sorbent copolymer preparation was incubated for 30 minutes at room temperature.

TABLE 22

| Time of incubation (min) | Titer of virus (TD50) |
|---|---|
| 60 | 10 (3.8) |
| 60* | 10 (5.2) |
| 240 | 10 (3.8) |
| 240* | 10 (4.5) |

*virus was incubated in the absence of sorbent copolymer preparation.

As shown by the results, the most effective anti-HIV activity of the sorbent copolymer preparation appears after incubating the virus and the sorbent copolymer for 30 min and 60 min. With these incubation times the TD₅₀ of HIV decreased to 1.2 and 1.4, respectively. When the incubation time was extended to 240 minutes at a room temperature, a natural decay of viral infectivity was observed, even in the absence of sorbent copolymer, and incubation for 240 minutes with sorbent copolymer did not produce further antiviral effect beyond that observed with the 30 and 60 minute intervals.

TABLE 23

| Incubation time (min) | Titer (TD₅₀) |
|---|---|
| 60 | 10 (4) |
| 30* | 10 (5.2) |
| 240 | 10 (4) |
| 60* | 10 (5.5) |

*virus was incubated in the absence of sorbent copolymer preparation.

As can be seen from the results of TABLE 23, an increase of the level of dilution of the initial suspension does not lead to the increase of its antiviral activity. It is necessary to note, that the observed effect may be related to the changes of growing properties of cells in the presence of relatively large particles of preparation, which remained in non-centrifuged suspension, and which as a consequence may cause the change in viral replication properties.

Based on these results it is reasonable to conclude that the sorbent copolymer reduces antiviral activity of laboratory strain HIV-1/VS, and is likely to reduce the antiviral activity of other HIV strains as well. The highest antiviral activity of any preparation was observed at 30 min and 60 min incubation of a virus with sorbent copolymer, which reduced TD₅₀ to 1.2 and 1.4, respectively.

### EXAMPLE 11

### Toxicity Testing

Toxicity of the sorbent copolymer was studied in accordance with the “Methodological Guidelines for the Determination of the Toxic Properties of Preparations Used in Veterinary Medicine and Animal Husbandry” as set forth in the handbook “Veterinary Preparations,” edited by A. D. Tret'yakov, 1988, as well as the requirements set forth in the State Pharmacopoeia of the USSR, 11th ed. (1987).

The acute toxicity of the sorbent copolymer was studied on white mice aged 2 to 2.5 months, weighing 18 to 22 g; on guinea pigs aged 3 to 4 months and weighing 450 to 500 g; and 4 month old rabbits weighing 2000 to 3000 g. The results are presented below in TABLE 24.

TABLE 24

| | | Dose | | No. of animals | |
|---|---|---|---|---|---|
| Animal | Route | mg/kg | cm³/animal | survived/died | Result |
| Guinea pig | Oral | 100 | 0.4 | 15/0 | nontoxic |
| | (capsules - 500 mg/4 cm³) | 500 | 2.0 | 15/0 | nontoxic |
| | | 1000 | 4.0 | 15/0 | nontoxic |
| | Intramuscular | 100 | 0.42 | 15/0 | nontoxic |
| | (ampules of 600 mg/5 cm³) | 200 | 0.84 | 15/0 | nontoxic |
| | | 500 | 2.1 | 15/0 | nontoxic |
| Rabbit | Oral | 100 | 1.6 | 9/0 | nontoxic |
| | (capsules - 500 mg/4 cm³) | 200 | 3.2 | 9/0 | nontoxic |
| | | 500 | 8.0 | 9/0 | nontoxic |
| | | 1000 | 15.0 | 9/0 | nontoxic |
| | Intramuscular | 100 | 1.7 | 9/0 | nontoxic |
| | (ampules of 600 mg/5 cm³) | 200 | 3.4 | 9/0 | nontoxic |
| | | 500 | 8.3 | 9/0 | nontoxic |
| | Intravenous | 100 | 1.7 | 9/0 | nontoxic |
| | (in ampules of 600 mg/5 cm³) | 200 | 3.4 | 9/0 | nontoxic |
| | | 400 | 6.8 | 9/0 | nontoxic |

Additionally, the toxicity of the sorbent copolymer was studied in 30-day-old chicks, 10-day-old ducklings, newborn and 2 to 6-month-old calves, and 3 to 5-year-old cows. For oral administration, the sorbent copolymer powder was suspended in 4 cm³ of water and the suspension packaged into 500-mg capsules. For intravenous and intramuscular administration, the sorbent copolymer was packaged into ampules as a suspension having a concentration of 120 mg/cm³. The results are presented in TABLE 25.

TABLE 25

| Species | | | | No. of | |
|---|---|---|---|---|---|
| and age | Route of | Dose | | animals | |
| of animal | administration | (mg/kg) | (cm³/animal) | tested | Result |
| Chicks | i.m. | 200 | 1.7 | 100 | nontoxic |
| 30 d/o | | | | | |
| 1 kg | | | | | |
| Ducklings | p.o. | 250 | 0.1 | 5 | nontoxic |
| 10 d/o | i.m. | 250 | 0.1 | 5 | nontoxic |
| 50-70 g | i.v. | 250 | 0.1 | 5 | nontoxic |
| Calves | p.o. | 40 | 8.0 | 3 | nontoxic |
| 2-3 d/o | i.m. | 10 | 2.0 | 3 | nontoxic |
| 25 kg | | | | | |
| Calves | p.o. | 40-50 | 20.0 | 8 | nontoxic |
| 2-3 m/o | | 100 | 40.0 | 10 | nontoxic |
| 50 kg | i.m. | 10 | 4.2 | 6 | nontoxic |
| | | 20 | 8.4 | 6 | nontoxic |
| | | 50 | 21.0 | 6 | nontoxic |
| | i.v. | 50 | 21.0 | 10 | nontoxic |
| Calves | p.o. | 100 | 80.0 | 5 | nontoxic |
| 4-6 m/o | i.m. | 50 | 42 | 5 | nontoxic |
| 100 kg | i.v. | 50 | 42 | 5 | nontoxic |
| Cows | i.m. and i.v. | 60 | 40 and 60 | 6 | nontoxic |
| 3-5 y/o | q.o.d | | | | |
| 300-400 | | | | | |
| kg | | | | | |

d/o = “days old”;
m/o = “weeks old”;
y/o = “years old”;
i.m. = “intramuscular”;
i.v. = “intravenous”;
p.o. = “oral”;
q.o.d = “every other day”

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. A method of treating an infection, the method comprising administering to a mammal in need thereof a compound of the formula xA₂O.ySb₂O₅.zSiO₂, wherein x is between 0.5 and 3, y is between 0.5 and 1.5, z is between 0.5 and 4; and A is H, Na, K, Rb, or Cs, and wherein the compound has a particle size range, said particle size range being between 0.1 and 100 μm.

2. The method of claim 1, wherein the route of administration is selected from the group consisting of oral and intramuscular.

3. The method of claim 2, wherein said infection is a viral infection.

4. The method of claim 3, wherein said viral infection is caused by rabies virus.

5. The method of claim 3, wherein said viral infection is Aujeszky's disease.

6. The method of claim 3, wherein said viral infection is caused by rotavirus.

7. The method of claim 3, wherein said viral infection is caused by bovine leucosis virus.

8. The method of claim 3, wherein said viral infection is caused by hepatitis C virus.

9. The method of claim 3, wherein said viral infection is caused by influenza virus.

10. The method of claim 9, wherein said influenza virus is Influenza A virus subtype H5N1.

11. The method of claim 3, wherein said viral infection is caused by a human immunodeficiency virus.
